(19) United States
(12) Patent Application Publication
(10) Pub. No.: US 2015/0025579 A1
(43) Pub. Date: Jan. 22, 2015

(54) POLYAXIAL BONE ANCHORING DEVICE

(71) Applicants: Lutz Biedermann, VS-Villingen (DE); Wilfried Matthis, Weisweil (DE)

(72) Inventors: Lutz Biedermann, VS-Villingen (DE); Wilfried Matthis, Weisweil (DE)

(21) Appl. No.: 14/335,747

(22) Filed: Jul. 18, 2014

Related U.S. Application Data
(60) Provisional application No. 61/856,517, filed on Jul. 19, 2013.

(30) Foreign Application Priority Data
Jul. 19, 2013 (EP) 13177279.0

(51) Int. Cl. A61B 17/70

(52) U.S. Cl. CPC A61B 17/7035; A61B 17/7011; A61B 17/7032
USPC 606/266; 606/279

(57) ABSTRACT

A polyaxial bone anchoring device including an anchoring element including a shank and a head; a receiving part to pivotably receive the head, the receiving part having a first end, a second end, a central axis extending between the first and second ends, a coaxial bore, and first and second recesses on each side of the coaxial bore for receiving a rod, a longitudinal axis of the recess being substantially perpendicular to the central axis; a first clamping part having a rod supporting surface extending along the longitudinal axis and in the first recess when in the receiving part, a center of the first clamping part along a direction transverse to the longitudinal axis including a groove at the rod supporting surface; a second clamping part to exert pressure on the rod and in the first recess when in the receiving part; and a locking device to secure the rod.

### CROSS-REFERENCE TO RELATED APPLICATIONS

The present disclosure claims the benefit of U.S. Provisional Patent Application Ser. No. 61/856,517, filed on Jul. 19, 2013, the contents of which are hereby incorporated by reference in their entirety, and claims priority from European Patent Application EP 13177279.0, filed on Jul. 19, 2013, the contents of which are hereby incorporated by reference in their entirety.

### BACKGROUND

1. Field of the Invention

The invention relates to a polyaxial bone anchoring device that includes an anchoring element with a head and a shank and a receiving part including a seat for pivotably receiving the head and a recess forming a channel to receive a rod therein. The polyaxial bone anchoring device further includes a first clamping part for supporting the rod in the recess from its lower side and a second clamping part for acting on the upper side of the rod. The first clamping part and the second clamping part both extend laterally into the channel provided by the recess. The polyaxial bone anchoring device further includes a locking device that is configured to exert pressure on the second clamping part. Such a polyaxial bone anchoring device is particularly suitable in connection with rods having a surface that is sensitive to corrosion, abrasion, and wear and in connection with curved rods.

2. Description of the Related Art

A polyaxial bone anchoring device wherein the rod is clamped between a pressure element having a recess for the rod and a set screw is known from US 2003/0100896 A1.

Various other solutions for clamping a specific type of rod for improving the fixation of such rods are known. For example, US 2008/0086132 A1 describes a polyaxial bone anchoring device for tubular rods. A filling piece is arranged between a locking element and the rod, wherein the filling piece comprises a rod contacting surface which contacts a portion of the rod and wherein the shape of the rod contacting surface is adapted to the shape of the portion of the rod.

Another bone anchoring device, according to US 2007/0093820 A1, has a filling piece between the rod and a clamping element, such as a threaded nut, wherein the filling piece includes transverse ribs to clamp a rod made of an elastomer material.

U.S. Pat. No. 7,338,491 B2 describes a locking mechanism configured to engage and lock a relative position of a bone fixation device and a relative position of a stabilization device with a locking element that has a rectangular shape and is longer than the width of the seat such that, when the locking element and the seat are engaged, the locking element protrudes from the side portion.

In particular, when using rods made fully or partially of Nitinol, a reduced contact area for clamping may lead to increased local forces and an increased notch effect or even plastic deformation of the rod or the clamping part.

There is still a need for a polyaxial bone anchoring device with an improved rod clamping design.

### SUMMARY

It is the object of the invention to provide a polyaxial bone anchoring device that has an improved rod clamping design so that it is suitable for a variety of rods.

The polyaxial bone anchoring device has first and second clamping parts that extend into the channel of the receiving part that accommodates the rod. Hence, a greater surface (e.g., a larger surface) for supporting and clamping the rod is provided. Due to the greater clamping surface, a notch effect is reduced and there is no or only little plastic deformation of the parts.

Furthermore, the clamping pressure is distributed over a greater area compared to clamping the rod with only a set screw.

Possible damage to the rod surface that may result in corrosion and, in the case of Nitinol rods, nickel release can be reduced or prevented.

The first and the second clamping parts can be slightly flexible so that they adapt to the surface of the rod when they are pressed against the rod.

Multiple contact areas provided by the first and the second clamping parts allow the use of curved rods as well as straight rods, for example, curved MIS rods (rods for minimally invasive surgery).

### BRIEF DESCRIPTION OF THE DRAWINGS

Further features and advantages of the invention will become apparent from the description of embodiments by means of the accompanying drawings. In the drawings:

FIG. 1 shows a perspective exploded view of a polyaxial bone anchoring device with a straight rod according to a first embodiment.

FIG. 2 shows a perspective view of the polyaxial bone anchoring device according to FIG. 1 in an assembled state with the rod clamped.

FIG. 3 shows a cross-sectional view of the polyaxial bone anchoring device according to FIG. 2, wherein the section is taken on a plane through a central axis of the receiving part and perpendicular to a rod axis.

FIG. 4a shows a cross-sectional view of the polyaxial bone anchoring device in an assembled state according to FIG. 2, wherein the section is taken on a plane including the central axis of the receiving part and the rod axis.

FIG. 4b shows a cross-sectional view of the polyaxial bone anchoring device as in FIG. 2 with a curved rod.

FIG. 5 shows a perspective view from a top of a first clamping part of the polyaxial bone anchoring device according to the first embodiment.

FIG. 6 shows a perspective view from a bottom of the clamping part of FIG. 5.

FIG. 7 shows a top view of the clamping part of FIG. 5.

FIG. 8 shows a cross-sectional view of the clamping part along the line D-D in FIG. 7.

FIG. 9 shows a side view of the clamping part of FIG. 5.

FIG. 10 shows a perspective view from a top of a second clamping part of the polyaxial bone anchoring device according to the first embodiment.

FIG. 11 shows a perspective view from a bottom of the clamping part according to FIG. 10.

FIG. 12 shows a top view of the clamping part according to FIG. 10.

FIG. 13 shows a cross-sectional view of the clamping part according to the line A-A in FIG. 12.

FIG. 14 shows a side view of the clamping part shown in FIG. 10.

FIG. 15 shows a perspective exploded view of a polyaxial bone anchoring device with a rod according to a second embodiment.

FIG. 16 shows a perspective view of the polyaxial bone anchoring device according to FIG. 15 in an assembled state with the rod clamped.

FIG. 17 shows a cross-sectional view of the polyaxial bone anchoring device according to FIG. 16, wherein the section is taken on a plane through the central axis of the receiving part and perpendicular to the rod axis.

FIG. 18 shows a cross-sectional view of the polyaxial bone anchoring device according to FIG. 16, wherein the section is taken on a plane through the central axis of the receiving part and includes the rod axis.

FIG. 19 shows a perspective view from a top of a first clamping part of the polyaxial bone anchoring device according to the second embodiment.

FIG. 20 shows a perspective view from a bottom of the clamping part of FIG. 19.

FIG. 21 shows a top view of the clamping part of FIG. 19.

FIG. 22 shows a cross-sectional view of the clamping part along the line C-C in FIG. 21.

FIG. 23 shows a side view of the clamping part of FIG. 19.

FIG. 24 shows a perspective view from a top of a second clamping part according to polyaxial bone anchoring device of the second embodiment.

FIG. 25 shows a perspective view from a bottom of the upper clamping part shown in FIG. 24.

FIG. 26 shows a top view of the clamping part of FIG. 24.

FIG. 27 shows a cross-sectional view of the clamping part along the line B-B in FIG. 26.

FIG. 28 shows a side view of the clamping part of FIG. 24.

FIG. 29 shows a perspective exploded view of a polyaxial bone anchoring device with a rod according to a third embodiment.

FIG. 30 shows a cross-sectional view of the polyaxial bone anchoring device according to FIG. 29, wherein the section is taken on a plane through the central axis of the receiving part and perpendicular to the rod axis.

### DETAILED DESCRIPTION

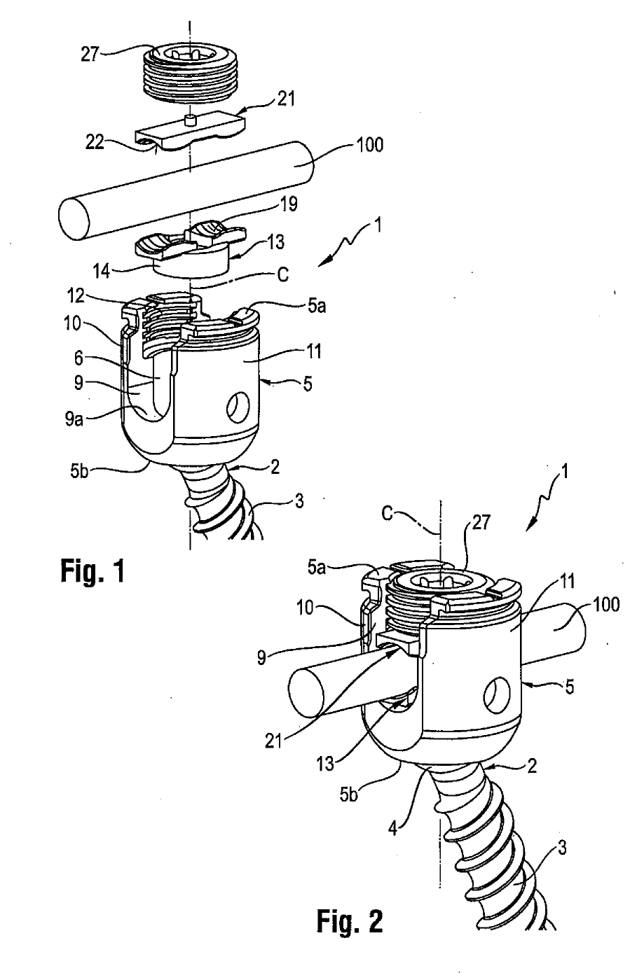

Referring to FIGS. 1 to 4, the polyaxial bone anchoring device 1 according to a first embodiment includes a bone anchoring element 2 in the form of a bone screw having a shank 3 with a threaded portion and a tip (not shown) and a head 4. In the embodiment shown, the head 4 has a spherical segment shape. A recess 4a for engagement with a tool is provided at a free end of the head 4.

The polyaxial bone anchoring device further includes a receiving part 5 that has a first end 5a and an opposite second end 5b, a central axis C extending through the planes defined by the first end 5a and the second end 5b, respectively, and a coaxial bore 6 extending from the first end 5a to a distance from the second end 5b. At the second end 5b, an opening 7 is provided, a diameter of which is smaller than a diameter of the bore 6. A spherically-shaped section 8 is provided adjacent to the opening 7 that forms a seat for the head 4. The section that forms the seat can have other shapes, for example, a conical shape, that allow the receiving part 5 to pivotably hold the head 4 therein.

The receiving part 5 includes a substantially U-shaped recess 9 that starts at the first end 5a and extends to a distance from the second end 5b for receiving a rod 100. The substantially U-shaped recess has a bottom 9a. Due to the U-shaped recess 9, two free legs 10, 11 are formed. On the legs 10, 11, an internal thread 12 is provided.

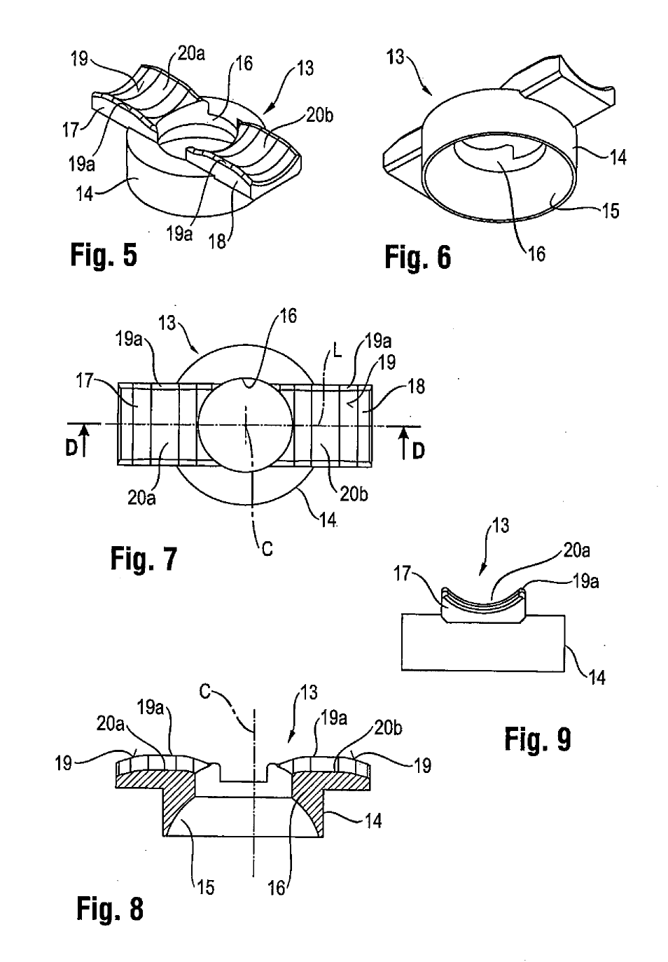

Referring now to FIGS. 5 to 9, the polyaxial bone anchoring device 1 further includes a first clamping part 13 supporting the rod 100 from a lower side of the rod. In the embodiment shown, the first clamping part 13 is a monolithic piece. The first clamping part 13 includes a first substantially cylindrical portion 14 with an outer diameter that is only slightly smaller than the inner diameter of the bore 6 to allow the first clamping part 13 to be moved in an axial direction of the bore 6 once it is inserted in the bore 6. On its lower side (e.g., a side facing towards the second end 5b of the receiving part in an installed state), the first clamping part 13 includes a spherical segment-shaped recess 15, the radius of which corresponds substantially to a radius of the head 4 of the anchoring element 2. The height of the cylindrical portion 14 in an axial direction is, as can be seen in particular in FIGS. 4a and 4b, such that when the first clamping part 13 rests on the head 4 of the anchoring element 2, the cylindrical portion 14 extends past the bottom 9a of the substantially U-shaped recess 9 of the receiving part 5 in the direction of the first end 5a.

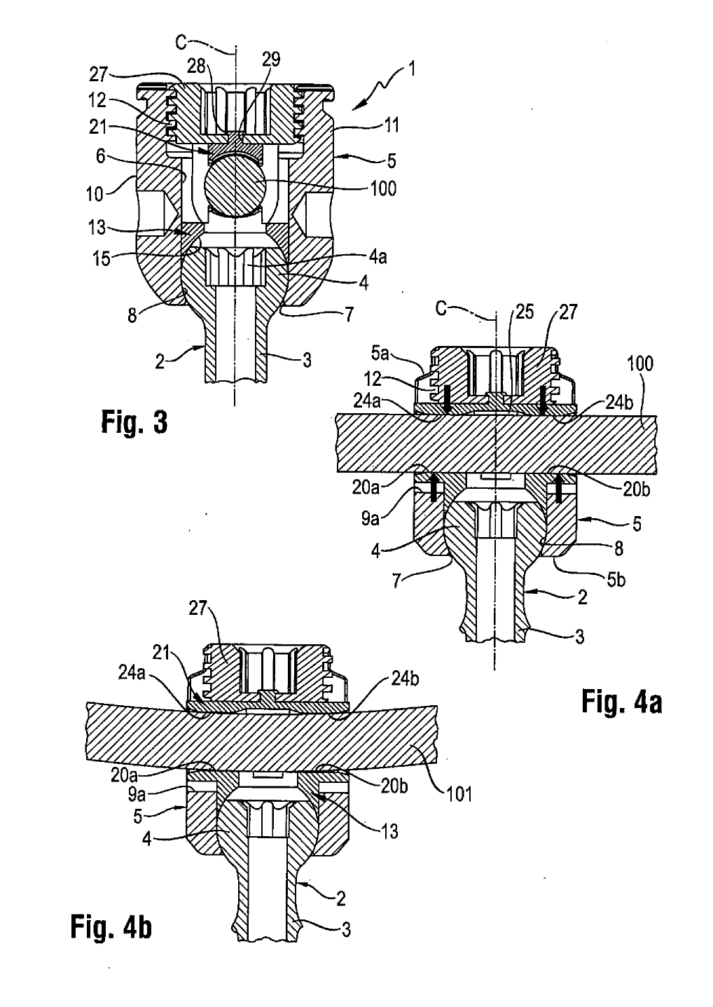

The first clamping part 13 further includes a coaxial bore 16 that allows access to the head 4 of the anchoring element with a tool when the anchoring element is held in the receiving part 5. On its side opposite to the spherical recess 15, the first clamping part 13 includes two laterally extending arms 17, 18 that are offset from each other by 180° and extend from the bore 16 in a direction perpendicular to the bore axis beyond the outer surface of the cylindrical portion 14. The arms 17, 18 have a substantially rectangular outer contour with a circular segment portion facing the coaxial bore 16 and having a corresponding diameter. An upper surface of the arms 17, 18 forms a rod supporting surface 19 that is substantially groove-shaped with a longitudinal axis L of the groove being perpendicular to the central axis C. As can be seen in particular in FIGS. 5 and 9, the shape of the groove is substantially a cylinder-segment with an inner diameter that is only slightly larger than a diameter of a corresponding cylinder segment of the rod 100, so that when the rod 100 is placed on the rod supporting surface 19 the rod fits into the groove as can be seen in FIG. 3.

Referring to FIGS. 7 and 8, the rod supporting surface 19 includes portions with different depths along the longitudinal axis L. Each arm has a portion 20a, 20b having a smallest depth, whereby two bulges 20a, 20b are formed that each decline smoothly in a longitudinal direction towards the coaxial bore 16 on one side and towards an outer free end of the arms 17, 18 on the other side. An upper surface of the bulges 20a, 20b may be even with each other. The steepness of the declining portions to the right and left of the bulges 20a, 20b in the longitudinal direction may be the same or may be different from each other. For example, the steepness may be greater towards the outer free end of the arms 17, 18 than towards the coaxial hole 16. A rim 19a of the rod supporting surface 19 varies in height corresponding to the portions of the arms and has a highest position at the bulges 20a, 20b. The bulges 20a, 20b have a width in the longitudinal direction that provides a contact area with the rod that is greater than a line or point contact.

The arms 17, 18 have such a size (e.g., a length) that they extend substantially up to the end of the channel formed by the U-shaped recess 9 in the receiving part. As can be seen in FIGS. 4a and 4b, the free ends of the arms 17, 18 are substantially flush with the outer surface of the receiving part 5. The width of the arms is only slightly smaller than the width of the U-shaped recess 9 of the receiving part 5, so that the arms are guided in the recess 9.

The rod supporting surface 19 provides an enlarged contact area for the rod compared to conventional pressure elements, the size of which is confined to the size of the bore 6. Due to the bulges 20a, 20b, two contact areas that are spaced apart from each other at a distance greater than the diameter of the bore 6 are provided. Also, the rounded design of the contact areas enlarges the possibilities of contact between the rod 100 and the first clamping part 13. Therefore, it is possible to also clamp rods that have curvature, such as curved MIS rods.

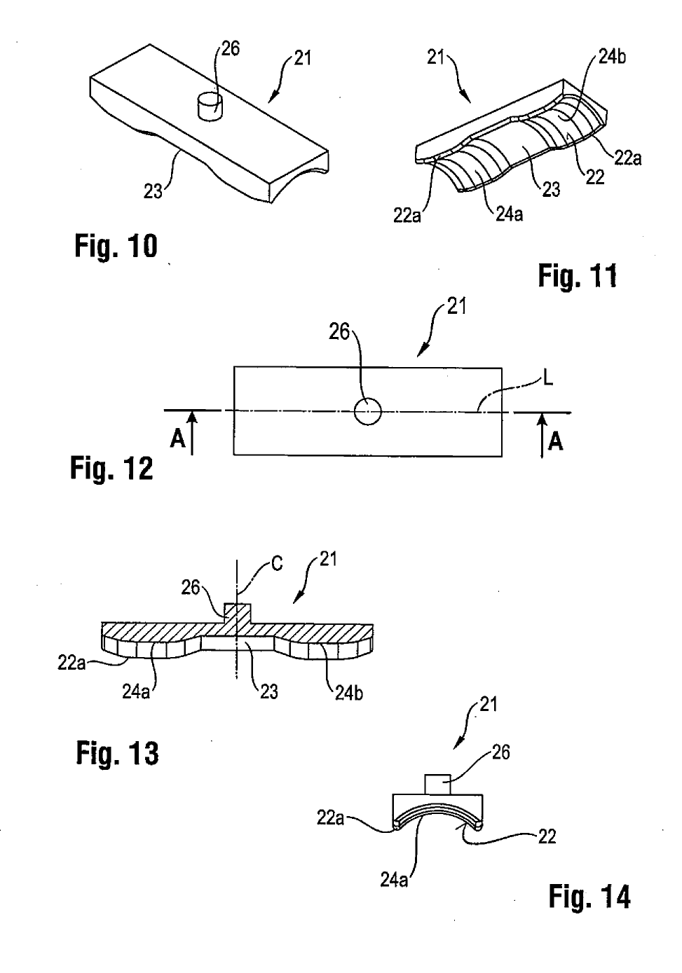

Referring now additionally to FIGS. 10 to 14, the polyaxial bone anchoring device also includes a second clamping part 21. As can be seen in FIG. 12, the second clamping part 21 has a substantially rectangular contour. A length of the second clamping part corresponds substantially to a length of the channel formed by the U-shaped recess 9 of the receiving part. A width of the second clamping part 21 is only slightly smaller than a width of the U-shaped recess 9 of the receiving part 5 so that the second clamping part 21 may be inserted into the U-shaped recess and be guided therein.

A lower side of the second clamping part 21 that faces the rod 100 when the second clamping part 21 is in the receiving part 5 is substantially groove-shaped with a width of the groove being substantially the same as a width of the groove of the first clamping part 13. The surface of the groove forms a rod facing surface 22 that is configured to come into contact with the rod and to exert pressure onto the rod 100 with at least a portion of the rod facing surface. The rod facing surface 22 includes a substantially flat central portion 23 and two bulges 24a, 24b on each side of the central portion 23. Similar to the first clamping part 13, the bulges 24a, 24b form shallow portions of the groove in the longitudinal direction. A top of each of the bulges 24a, 24b may be even with each other. The bulges 24a, 24b decline smoothly towards free ends of the second clamping part 21 and towards the center portion of the second clamping part 21 with the same or with different steepnesses. A rim 22a of the rod facing surface 22 varies in height corresponding to the portions 22, 24a, 24b of the rod facing surface and has a highest position at the position of the bulges 24a, 24b.

A distance between the bulges 24a and 24b of the second clamping part 21 can be the same as the distance between the bulges 20a and 20b of the first clamping part 13. The distance may also be different.

As can be seen in FIGS. 4a and 4b, because the rim 22a of the rod facing surface 22 is lower in the central portion 23, a gap 25 between the rod 100 and the rod contacting surface 22 is formed.

On its side facing away from the rod facing surface 22, the second clamping part 21 includes a projection 26 that is cylindrical and serves to rotationally engage a locking screw 27 as shown in FIGS. 3 and 4. The second clamping part 21 is rotatably coupled to the locking screw 27 that cooperates with the internal thread 12 of the legs 10, 11 of the receiving part 5. As depicted in FIG. 3, the second clamping part 21 may have a rim 28 at the end of the cylindrical projection 26 that holds the second clamping part 21 in a bore 29 at a lower side of the locking screw 27. An upper side of the second clamping part 21 that faces towards the locking screw 27 is substantially even (e.g., substantially flat).

All parts of the bone anchoring device are made from a body compatible material, such as a body compatible metal or metal alloy or a body compatible plastic material. Examples of such metals or metal alloys are titanium, stainless steel, and titanium alloys, such as, for example, Nitinol. An example of a body compatible plastic is PEEK (Polyetheretherketone).

At least two bone anchoring devices and a rod form a spinal stabilization system. The rods to be used are preferably rods that have a more sensitive surface or rods that may cause problems if used with conventional bone anchoring devices. In particular, the polyaxial bone anchoring device can be used with Nitinol rods. Another type of rod that can be used are curved rods, in particular, curved MIS rods.

In use, the first clamping part 13 may be pre-assembled with the receiving part 5 and the anchoring element 2. The first clamping part 13 can be inserted into the receiving part 5 by orientating it such that the arms 17, 18 extend into the U-shaped recess 9. The second clamping part 21 may be pre-assembled with the locking screw 27. First, two polyaxial bone anchoring devices, each including the pre-assembled receiving part 5, anchoring element 2, and first clamping part 13, are inserted into two bone parts or two vertebrae, respectively. Thereafter, the rod 100 is inserted into the U-shaped recess 9 of the receiving part 5 until it rests with its lower side on the bulges 20a, 20b of the rod supporting surface 19. Then, the pre-assembled locking screw 27 and first clamping part 21 is inserted into the receiving part until the rod facing surface 22 contacts the rod 100 at the contact areas provided by the bulges 24a, 24b. Due to the rotatable connection, the locking screw 27 can be tightened while the second clamping part 21 engages the rod 100. When the locking screw 27 is tightened, pressure is exerted via the second clamping part 21 onto the rod 100 and from the rod 100 onto the first clamping part 13. The first clamping part 13 also exerts pressure onto the head 4. The pressure exerted onto the rod 100 is distributed over a larger area compared to conventional devices. Furthermore, the pressure is exerted via multiple contact areas, in particular, via four contact areas as shown in FIGS. 4a and 4b. The contact areas are provided by uppermost portions of the bulges 20a, 20b, 24a, 24b. In the embodiment shown, the contact areas of the rod supporting surface 19 and the rod facing surface 22 lie at the same longitudinal positions along an axis of the rod. Final tightening of the locking screw 27 also locks the head 4 in an angular position due to the pressure exerted onto it by the first clamping part 13.

Due to the enlarged contact area, a notch effect that may lead to corrosion and abrasion and the setting free of nickel particles may be reduced or even prevented.

While FIG. 4a shows the bone anchoring device with a straight rod 100, FIG. 4b shows the bone anchoring device with a curved rod 101. The smooth shape of the bulges 20a, 20b, 24a, 24b allows the clamping parts to adapt the contact area to the shape of the rod.

It shall be understood that in a modified first embodiment, more than two contact areas on each of the clamping parts 13, 21 may be provided. Also, the contact areas of the first clamping part 13 and the contact areas of the second clamping part 21 may have a different distance from each other so that the rod is clamped at different positions in a longitudinal direction from below and from above.

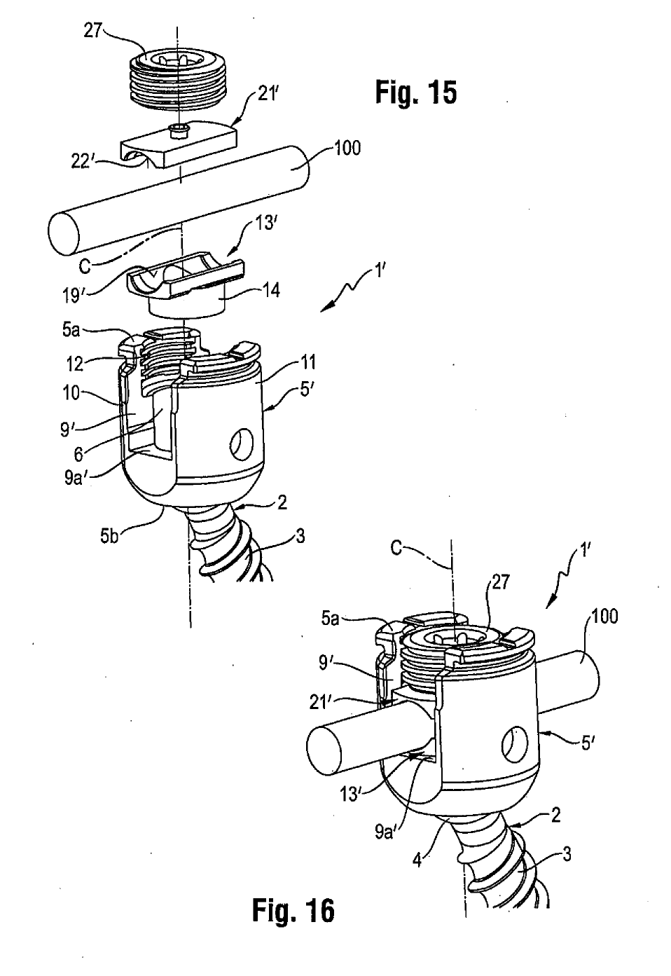

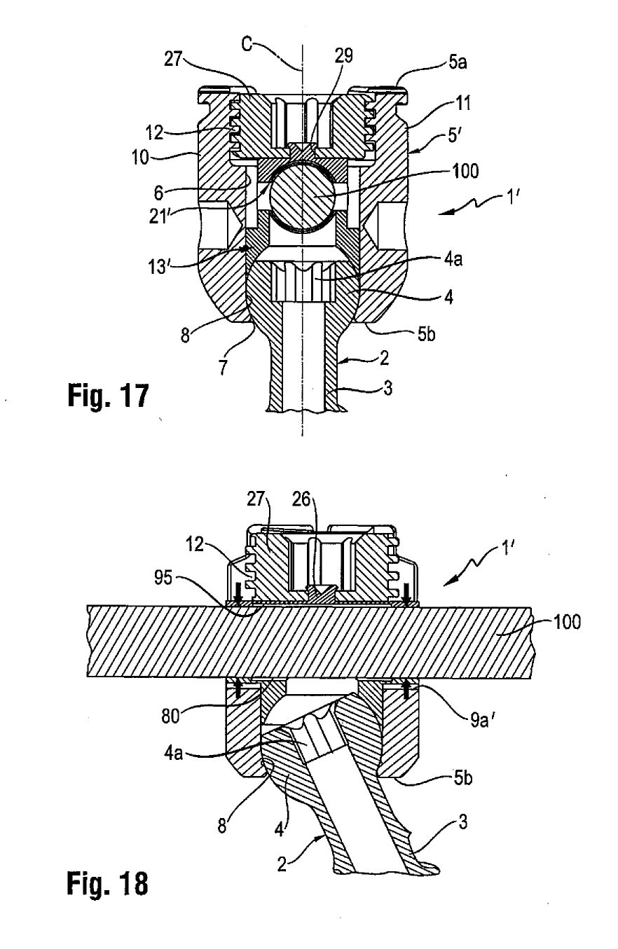

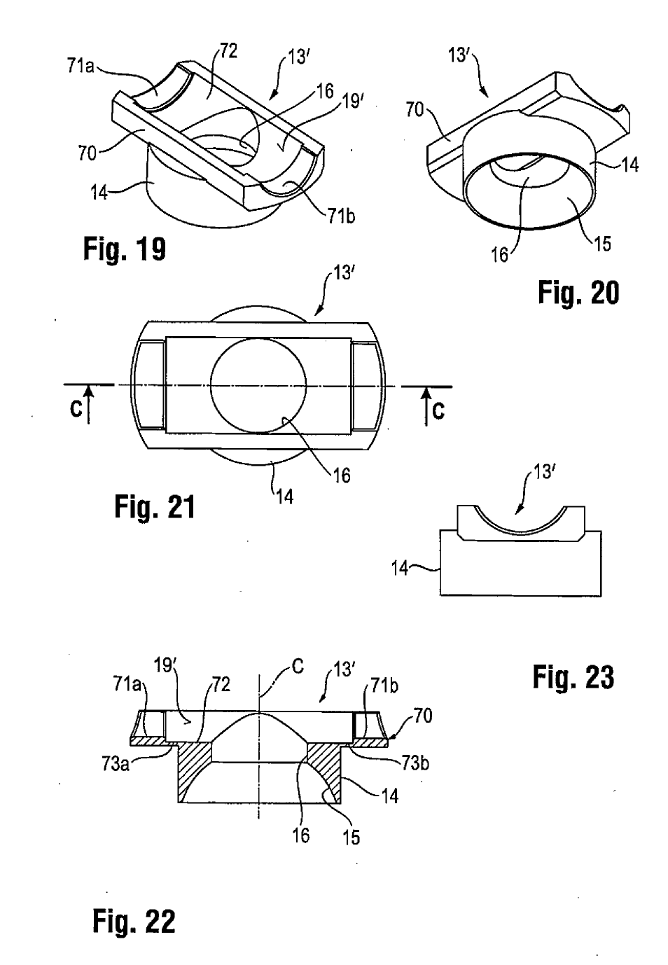

A second embodiment of the polyaxial bone anchoring device will be described with reference to FIGS. 15 to 18. The polyaxial bone anchoring device 1′ differs from the polyaxial bone anchoring device 1 according to the first embodiment mainly by the design of the first and the second clamping part. All other parts are the same or identical to the first embodiment and the parts and portions that are the same or highly similar to that of the first embodiment have the same references numerals and the description thereof will not be repeated.

The receiving part 5′ has in this embodiment a recess 9′ for receiving the rod with a substantially rectangular contour and a flat bottom 9a′.

The first clamping part 13′ includes the cylindrical portion 14 with the spherically-shaped recess 15 at an underside thereof and the coaxial bore 16. The first clamping part 13′ differs from the first clamping part 13 of the first embodiment only in a rod supporting surface 19′. The rod supporting surface 19′ is formed on a groove-like portion 70 that is provided at a side of the cylindrical portion 14 opposite to the spherically-shaped recess 15. The groove-like portion 70 has a substantially rectangular contour with rounded short sides, as shown in FIG. 21. A total length in the longitudinal direction along the longitudinal axis L of the groove-like portion 70 corresponds substantially to the axial length of the recess 9′ in the receiving part 5′ measured perpendicularly to the central axis C. A width the groove portion 70 is only slightly smaller than a width of the recess 9′ in the receiving part 5′ and allows the groove-like portion 70 to be placed between the legs 10, 11 that are separated by the recess 9′.

The rod supporting surface 19′ includes, on each outer end of the groove-like portion 70, a shallow part 71a, 71b and a deeper part 72 therebetween. Hence, the shallow parts form slight prominences 71a, 71b that may have an even upper surface with each other. Between the prominences 71a, 71b and the cylindrical portion 14, the groove-like portion 70 has thinner bottom portions 73a, 73b, as can be seen in FIG. 22. This renders the groove-like portion 70 slightly flexible. As can be seen in particular in FIG. 18, there is a gap 80 between the rod supporting surface 19′ and the rod 100 in the shallower region 72 when the rod 100 rests on the prominences 71a, 71b. The gap 80 allows the groove-like portion 70 to adapt to the surface of the rod when pressure is exerted via the rod onto the rod supporting surface 19′ of the first clamping part 13′.

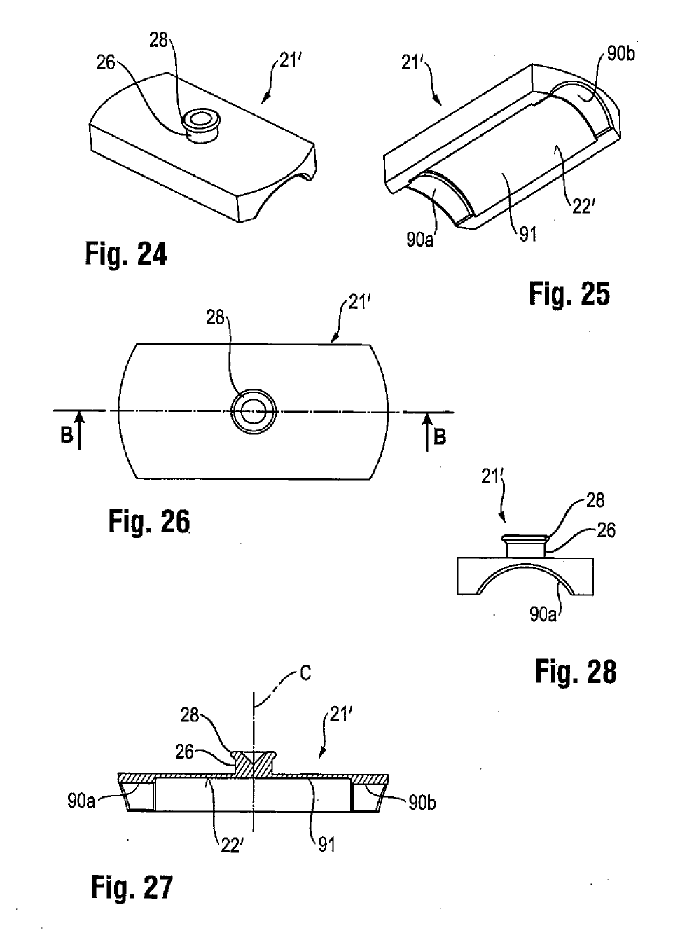

Referring further to FIGS. 24 to 27, the second clamping part 21′ has an outer contour corresponding to that of the groove-like portion 70 of the first clamping part 13′. The second clamping part 21′ includes a rod facing surface 22′ that has a shape corresponding to the rod supporting surface 19′ of the first clamping part 13′. That is, the rod facing surface 22′ has a groove-like shape with prominences 90a, 90b at either end of the second clamping part 21′ that form rod contact areas and a cylinder-segment shaped recess 91 between the prominences 90a, 90b.

The second clamping part 21′ is rotatably connected to the locking screw 27 as in the first embodiment. An upper surface of the second clamping part 21′ that faces the locking element 27 is substantially even (e.g., substantially flat) in order to allow transfer of pressure from the locking element 27 evenly onto the second clamping part 21′.

As can be seen in FIG. 18, the recess 91 leads to a gap 95 between the rod 100 and the rod facing surface 22′. This gap 95 renders the second clamping part 21′ slightly flexible and allows the rod facing surface 22′ to adapt to the surface of the rod 100.

In use, when the locking screw 27 is tightened, the pressure exerted by the locking screw 27 is transferred via the second clamping part 21′ and the rod 100 onto the first clamping part 13′. Preferably, the rod contacting areas are positioned at the same locations in an axial direction along the rod axis on the lower side and the upper side of the rod 100. Because the groove-like portion 70 of the first clamping part 13′ and the second clamping part 21′ are slightly flexible, they can adapt to the surface of the rod. Hence, the clamping area can be enlarged, resulting in improved clamping without the risk of notch effects and plastic deformation, as well as reducing the risk of abrasion and corrosion. When the rod presses onto the rod supporting surface 19′, the first clamping part 13′ exerts a pressure onto the head 4 that locks the head 4 in a specific angular position.

It shall be understood that the second embodiment may also be used with curved rods, because of the ability of the clamping parts to adapt to a surface of the rod. Furthermore, the position and number of the prominences is not limited to the position and number of the prominences as shown in this specific embodiment. There may be more than two contact areas. The contact areas may also be positioned away from the outer ends of the first and second clamping parts.

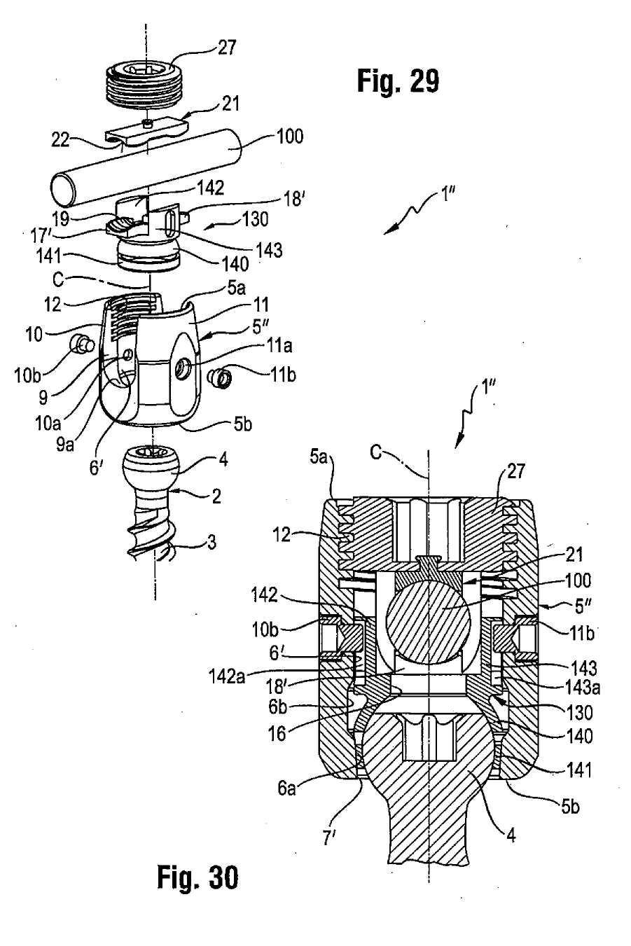

Referring to FIGS. 29 and 30, a third embodiment of the polyaxial bone anchoring device is described. The polyaxial bone anchoring device according to the third embodiment differs from the polyaxial bone anchoring device according to the first embodiment in that it is a bottom loading polyaxial bone anchoring device, i.e., the bone anchoring element 2 is configured to be introduced from the bottom end 5b of a receiving part 5″. The receiving part 5″ includes a central coaxial bore 6′ as in the first embodiment and an opening 7′ at the second end 5b that has a diameter greater than a largest diameter of the head 4, so that the head 4 can be inserted into the receiving part 5″ from the second end 5b. Adjacent to the opening 7′, there is a tapered section 6a that tapers towards the opening 7′. Between the tapered section 6a and the central coaxial bore 6′, the receiving part 5″ includes an enlarged bore section 6b having a greater diameter than that of the central bore 6′. The enlarged bore section serves to provide a space for a first clamping part 130 to expand therein. At about a center of each of the legs 10, 11 of the receiving part 5″, there is a through hole 10a, 11a that extends transverse to the central axis C and accommodates pins 10b, 11 b, respectively. The pins 10b, 11 b are configured to extend, in an inserted state, into the central coaxial bore 6′.

The first clamping part 130 is a monolithic piece. It includes a first cap-like portion 140 which is flexible, for example, due to a slit ring portion 141 that is part of the cap-like portion. The flexibility can be achieved through various other structures, such as, for example, a plurality of longitudinal and/or transverse slits. The size of the cap-like portion 140 is such that the head 4 of the bone anchoring element 2 can be inserted therein and is held therein in a frictional manner. As shown in FIG. 30, the cap-like portion 140 extends over a region of the head 4 having the largest diameter thereof. An outer surface of the cap-like portion 140 at the lower end thereof may be tapered and is configured to cooperate with the tapered section 6a of the receiving part 5″.

The first clamping part 130 also includes a coaxial bore 16 that allows access to the head 4 of the anchoring element with a tool. The first clamping part 130 further includes two laterally extending arms 17′, 18′ that are offset from each other by 180° and extend from the bore 16 in a direction perpendicular to the bore axis beyond the outer surface of the cap-like portion 140. The arms 17′, 18′ have substantially the same shape as the arms 17, 18 of the first clamping part 13 of the first embodiment. An upper surface of the arms 17′, 18′ forms the rod supporting surface 19 as in the first embodiment.

At 90° offset in both directions from the arms 17′, 18′, there are two upstanding legs 142, 143 with free ends at sides opposite to the cap-like portion 140. At an outer surface of the upstanding legs 142, 143 a recess 142a, 143a is provided that is elongated in a direction parallel to the central axis C. The elongated recesses 142a, 143a are configured to accommodate a front portion of the pins 10b, 11b. A bottom end of the elongated recesses 142a, 142b forms a stop for the first clamping part 130 when the first clamping part 130 is moved in the bore 6′ towards the first end 5a of the receiving part 5″, and the pins 10b, 11b abut against respective bottom ends of the recesses.

The second clamping part 21 is the same as in the first embodiment.

In use, the first clamping part 130 may be preassembled with the receiving part 5″. The arms 17′, 18′ extend from the bore 6′ into the recess 9. The bone anchoring element 4 is inserted from the second end 5b into the receiving part. In the insertion position, the first clamping part is moved towards the first end 5a until the pins 10b, 11b abut against the bottoms of the elongated recesses 142a, 142b. When the head 4 is inserted into the cap-like portion 140, the cap-like portion 140 expands into the enlarged bore section 6b of the receiving part 5″. When the rod 100 is inserted, it rests on the arms 17′, 18′ of the first clamping part 130. Then the second clamping part 21, which is mounted to the locking screw 27, is inserted into the receiving part and the locking screw 27 is tightened. Thereby, the rod 100 is clamped between the arms 17′, 18′ and the second clamping part 21 and the pressure exerted onto the first clamping part 130 moves the first clamping part 130 downward towards the second end 5b. The cap-like portion 140 of the first clamping part 130 is gradually compressed in the tapered portion 6a of the receiving part 5″ so that the head 4 is clamped in the cap-like portion 140 and prevented from falling out through the opening 7′. The final tightening of the locking screw 27 locks the bone anchoring element 2 in a specific angular position with respect to the central axis C. The effects on the rod and the rod surface that is clamped between the first and the second clamping part are the same as in the previous embodiments. Curved rods can also be used.

Many other modifications of the embodiments may be contemplated. For example, the bone anchoring devices shown in the embodiments are not limited to use together with solid rods. They may also be used together with tubular rods. Also, flexible rods may be contemplated, for example, elastomer rods.

Various modifications of the receiving part may be contemplated as well. For example, other designs of a top loading or a bottom loading polyaxial anchoring devices that include a pressure element for clamping the head can be used for the receiving part.

Other modifications of the locking device are possible. The locking device may include an outer nut or a cap. The connection of the locking element and the receiving part is not limited to a threaded connection.

The first clamping part has been described to include a portion that acts on the head 4 of the anchoring element. It may be contemplated that the portion acting on the head is an element separate from the rod supporting portion. The length of the first clamping part and the second clamping part in the longitudinal direction of the U-shaped recess may be smaller than the length of the recess but should be greater than the diameter of the coaxial bore of the receiving part.

A number of different embodiments are disclosed herein. It is appreciated that the different components and methods disclosed in the different embodiments can be mixed and matched to produce other, different embodiments.

1. A polyaxial bone anchoring device comprising:
an anchoring element comprising a shank and a head;
a receiving part configured to pivotably receive the head, the receiving part having a first end, a second end opposite to the first end, a coaxial bore, a first recess and a second recess on each side of the coaxial bore and adjacent to the first end for receiving a rod, and a central axis extending between the first end and the second end, the receiving part comprising two legs separated by the coaxial bore and the first and second recesses, a longitudinal axis of the first and second recesses being substantially perpendicular to the central axis;
a first clamping part having a rod supporting surface extending generally along the longitudinal axis perpendicular to the central axis and in the first recess when assembled in the receiving part, a center of the first clamping part along a direction transverse to the longitudinal axis of the rod supporting surface comprising a groove at the rod supporting surface;
a second clamping part configured to exert pressure on the rod when the rod is in the first recess and extending from the coaxial bore into the first recess when assembled in the receiving part; and
a locking device configured to exert pressure on the second clamping part to secure the rod.

2. The polyaxial bone anchoring device of claim 1, wherein the rod supporting surface extends from the coaxial bore into the second recess.

3. The polyaxial bone anchoring device of claim 1, wherein the first clamping part comprises a projection on the rod supporting surface outside the coaxial bore.

4. The polyaxial bone anchoring device of claim 3, wherein the first clamping part further comprises a plurality of projections on the rod supporting surface, ones of the plurality of projections being in the respective first and second recesses and separated along the longitudinal axis of the rod supporting surface by a depression.

5. The polyaxial bone anchoring device of claim 4, wherein the projections are bulges that are rounded in a longitudinal direction of the rod supporting surface.

6. The polyaxial bone anchoring device of claim 1, wherein the first clamping part further comprises a coaxial bore that is coaxial to the central axis.

7. The polyaxial bone anchoring device of claim 1, wherein the first clamping part further comprises a first arm and a second arm, the first and second arms extending from the coaxial bore of the receiving part into the first and second recesses, respectively, and
wherein each of the first and second arms have a portion of the rod supporting surface.

8. The polyaxial bone anchoring device of claim 7, wherein each portion of the rod supporting surface comprises a projection and tapers from the respective projection toward a free end and an opposite end of the respective first and second arms.

9. The polyaxial bone anchoring device of claim 1, wherein a portion of the rod supporting surface has a thickness in a direction perpendicular to an extension direction of the rod supporting surface less than a thickness of an adjacent portion of the rod supporting surface such that it is flexible.

10. The polyaxial bone anchoring device of claim 1, wherein the first clamping part further comprises a first portion that is configured to exert pressure on the head.

11. The polyaxial bone anchoring device of claim 10, wherein the first portion of the first clamping part has a recess at a side opposite to the rod supporting surface and the recess is configured to accommodate a portion of the head.

12. The polyaxial bone anchoring device of claim 10, wherein the first portion of the first clamping part is a flexible cap and is configured to expand to accommodate the head therein.

13. The polyaxial bone anchoring device of claim 1, wherein the second clamping part has a rod facing surface having a longitudinal axis perpendicular to the central axis.

14. The polyaxial bone anchoring device of claim 13, wherein the second clamping part comprises a plurality of projections on the rod facing surface that are separated along the longitudinal axis of the rod facing surface by a depression.

15. The polyaxial bone anchoring device of claim 14, wherein ones of the plurality of projections of the second clamping part are in the first and second recesses, respectively.

16. The polyaxial bone anchoring device of claim 15, wherein the rod facing surface of the second clamping part tapers from each of the projections toward the depression and respective free ends of the second clamping part.

17. The polyaxial bone anchoring device of claim 1, wherein respective free ends of the first clamping part and the second clamping part extend into the first recess such that the free ends are flush with an outer surface of the receiving part.

18. The polyaxial bone anchoring device of claim 1, wherein the second clamping part is configured to be rotatably coupled to the locking device.

19. The polyaxial bone anchoring device of claim 1, wherein respective free ends of the first clamping part and the second clamping are rounded in a direction perpendicular to the central axis.

20. A spinal stabilization system comprising at least two polyaxial bone anchoring devices according to claim 1 and a rod,
wherein the rod comprises a nickel-titanium alloy, and
wherein the rod is curved.

21. A method of attaching a rod to a bone via a polyaxial bone anchoring device, the bone anchoring device comprising an anchoring element comprising a shank and a head; a receiving part configured to pivotably receive the head, the receiving part having a first end, a second end opposite to the first end, a coaxial bore, a first recess and a second recess on each side of the coaxial bore and adjacent to the first end for receiving a rod, and a central axis extending between the first end and the second end, the receiving part comprising two legs separated by the coaxial bore and the first and second recesses, a longitudinal axis of the first and second recesses being substantially perpendicular to the central axis; a first clamping part having a rod supporting surface extending generally along the longitudinal axis perpendicular to the central axis and in the first recess when assembled in the receiving part, a center of the first clamping part along a direction transverse to the longitudinal axis of the rod supporting surface comprising a groove at the rod supporting surface; a second clamping part configured to exert pressure on the rod when the rod is in the first recess and extending from the coaxial bore into the first recess when assembled in the receiving part; and a locking device configured to exert pressure on the second clamping part to secure the rod, the method comprising:
inserting the anchoring element into a bone;
adjusting an angular position of the receiving part relative to the anchoring element when the head is in the receiving part;
inserting a rod into the coaxial bore of the receiving part; and
advancing the locking element in the coaxial bore towards a bottom end of the receiving part to advance the second clamping part toward the rod to secure the rod against the first clamping part.

22. The method of claim 21, further comprising assembling the first clamping part to the receiving part prior to the inserting of the anchoring element.

23. The method of claim 22, further comprising coupling the anchoring element to the receiving part prior to the inserting of the anchoring element.

24. The method of claim 21, further comprising assembling the first clamping part and the receiving part; and
coupling the anchoring element with the receiving part after the inserting of the anchoring element.

25. The method of claim 21, further comprising assembling the second clamping part and the locking element prior to the advancing of the locking element.
